(19) United States
(12) Patent Application Publication
(10) Pub. No.: US 2011/0137257 A1
(43) Pub. Date: Jun. 9, 2011

(54) Inserter for Transcutaneous Device

(75) Inventors: Steffen Gyrn, Ringsted (DK); Orla Mathiasen, Soro (DK)

(73) Assignee: UNOMEDICAL A/S

(21) Appl. No.: 13/000,471

(22) PCT Filed: Jul. 3, 2009
(86) PCT No.: PCT/EP09/58379

§ 371 Date: Feb. 14, 2011

Related U.S. Application Data
(60) Provisional application No. 61/134,144, filed on Jul. 7, 2008.

(51) Int. Cl. A61M 25/02

(52) U.S. Cl. 604/180; 604/174

(57) ABSTRACT

The present application relates to a device comprising a transcutaneous device and an inserter which transcutaneous device has a mounting surface. The device is upon delivery arranged in a sterile packing which packing functions as inserter and is attached to the transcutaneous device i.e. the packing and the inserter is one integrated unit. A device comprises a transcutanous device and an inserter. The transcutaneous device comprises—a mounting part (1) having a mounting surface (2),—a hard cannula (9)—a fluid path leading fluid from a fluid source to the hard cannula (9), The inserter comprises—a packing (10) at least partly covering the transcutaneous device before use which packing (10) comprises a non-deformable portion which portion cannot be deformed by the user during normal use and a deformable portion (10a) which portion can be reduced lengthwise in the direction of insertion during use. Preferably the mounting surface (2) is at least partly adhesive.

### TECHNICAL FIELD

The present invention relates to a device comprising a transcutaneous device and an inserter which transcutaneous device has a mounting surface. The device is upon delivery arranged in a sterile packing which packing functions as inserter and is attached to the transcutaneous device i.e. the packing and the inserter is one integrated unit.

### PRIOR ART

WO 2007/141210 discloses an assembly comprising a skin mountable device and a packing for this device. The packing of this device can be attached to the enclosed skin mountable device and then the packing can be used as an applicator for mounting the skin mountable device on the skin surface of the patient. When the packing is pushed in the direction of the housing i.e. along the skin surface, the packing will become detached from the skin mountable device. A body portion comprises a housing portion in which a cannula inserting mechanism is arranged i.e. the cannula inserting mechanism is not part of the packing and the cannula inserting mechanism according to this document is complex and comprises a coil-formed tension spring.

### SUMMARY OF THE INVENTION

The present invention relates to a device comprising a transcutaneous device and an inserter. The transcutaneous device comprises a mounting part having a mounting surface, a hard cannula, a fluid path leading fluid from a fluid source to the hard cannula. The inserter comprises or is constituted of a packing at least partly covering the transcutaneous device before use which packing comprises a non-deformable portion which portion cannot be deformed by the user during normal use and a deformable portion which portion can be reduced lengthwise in the direction of insertion during use.

That the inserter and the packing is constituted by the same piece(s) of material means that the amount of waste will be reduced and there will be fewer units for the user to handle.

According to one embodiment the mounting surface is at least partly adhesive and normally the mounting surface will be covered by a release liner before use in order to protect the adhesive surface(s). That the mounting surface is at least partly adhesive means that it might only be selected areas on the mounting surface which is provided with adhesive.

According to one embodiment the release liner can constitute a part of the packing covering and adhesive surface of the transcutaneous device before use.

According to one embodiment a hard plate is attached to the mounting part and this hard plate can be provided with a linear part of flexible material dividing the hard plate into a first part and a second part where the second part is fixed to the mounting part and the first part can pivot around an axis a formed by the linear flexible material. The hard plate can also have areas of soft flexible material e.g. around the insertion point of the cannula.

According to this embodiment an angle a between the first part and the second part in a first position can be smaller than 90 degrees and larger than the angle defined by the length of the hard cannula (9) which is to be inserted.

According to one embodiment the hard cannula can be made of steel or hard plastic.

According to one embodiment the fluid source is a reservoir attached to the mounting part.

According to one embodiment the packing can be formed of a hard deformable plastic e.g. of two or more types of hard plastic having different ability to be compressed i.e. having the extent of the material reduced.

According to one embodiment the mounting part can be provided with two at least partly adhesive surfaces, one surface facing the inserter packing and the mounting surface. This makes it possible place and adhere the device to the patients skin and then afterwards adhere the transcutaneous device to the opposite surface of the mounting part.

According to one embodiment the reservoir can contain insulin.

### DETAILED DESCRIPTION OF THE INVENTION

Embodiments of the invention will now be described with reference to the figures in which:

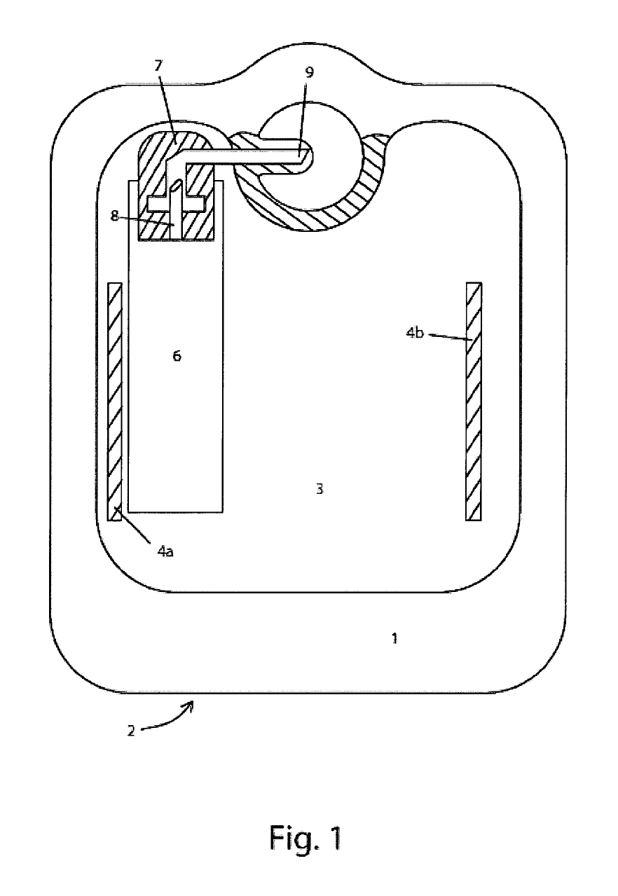

FIG. 1 shows an embodiment of a transcutaneous device seen from above after it has been positioned on the patients skin.

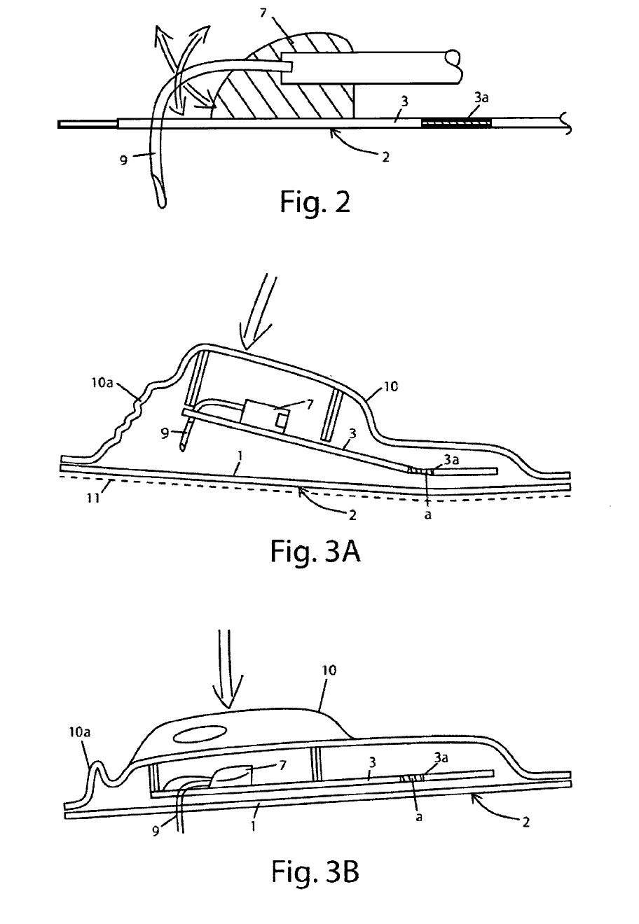

FIG. 2 shows an enlargement of a cannula in a second embodiment of a transcutaneous device seen from the side.

FIGS. 3a and 3b shows a cannula of FIG. 2 placed in a transcutaneous device in a first position (FIG. 3a) before insertion of the cannula and a second position (FIG. 3b) after insertion of the cannula.

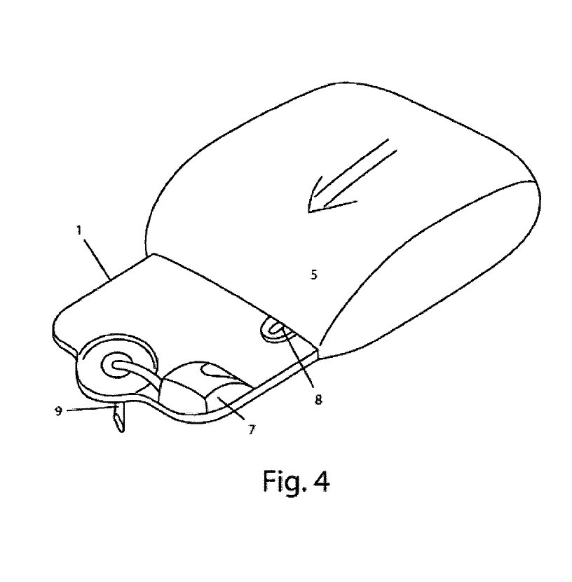

FIG. 4 shows a transcutaneous device placed on a patients skin having a pumping part partly mounted.

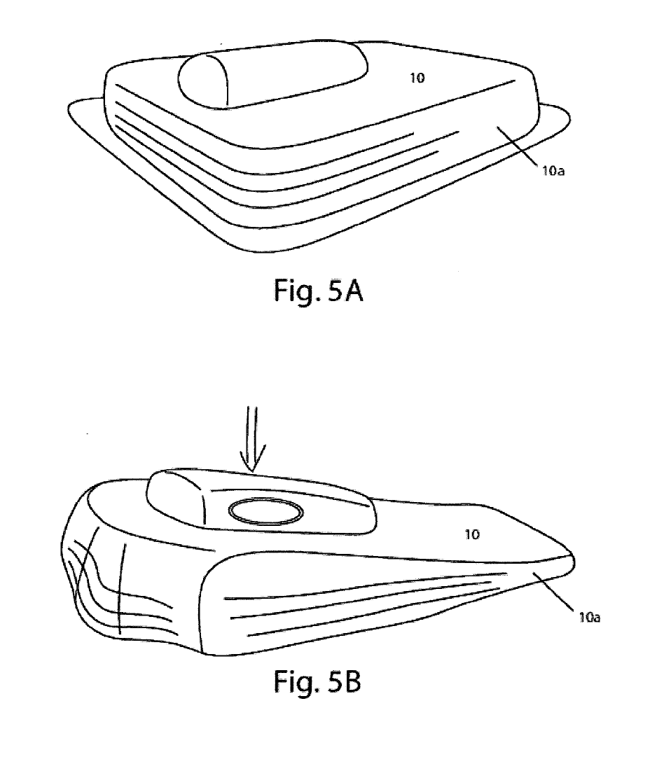

FIGS. 5a and 5b show a second embodiment of a packing in a first position (FIG. 5a) before insertion of the cannula and a second position (FIG. 5b) after insertion of the cannula.

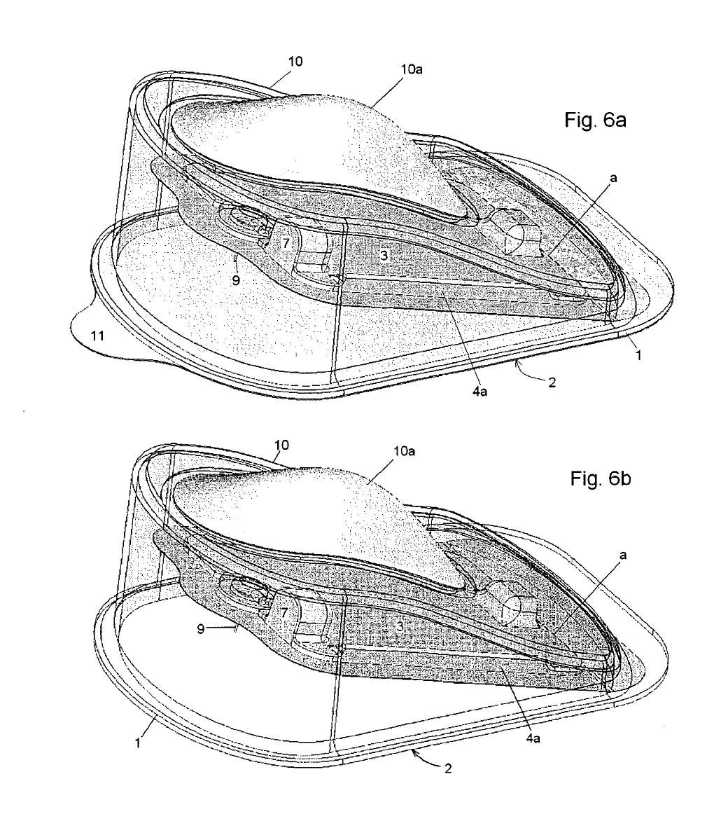

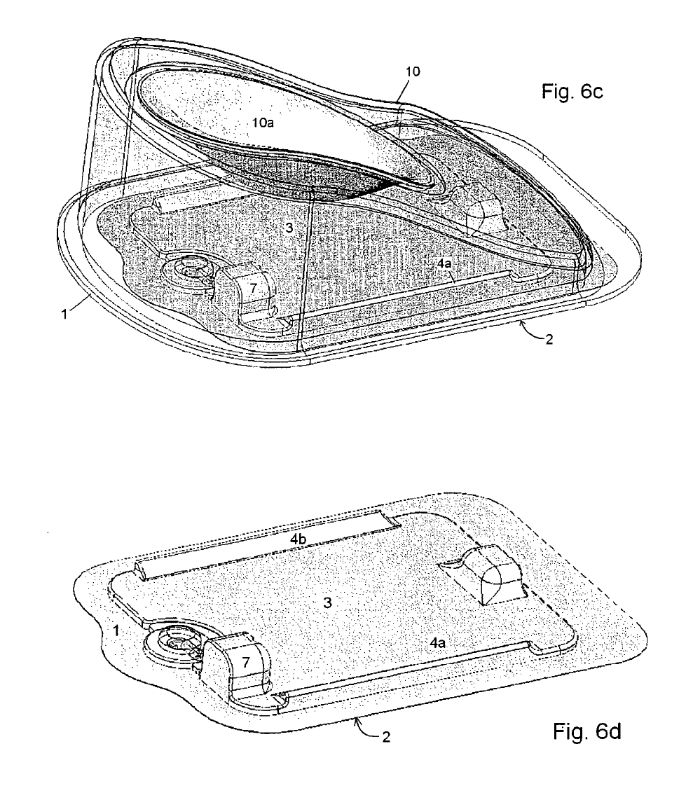

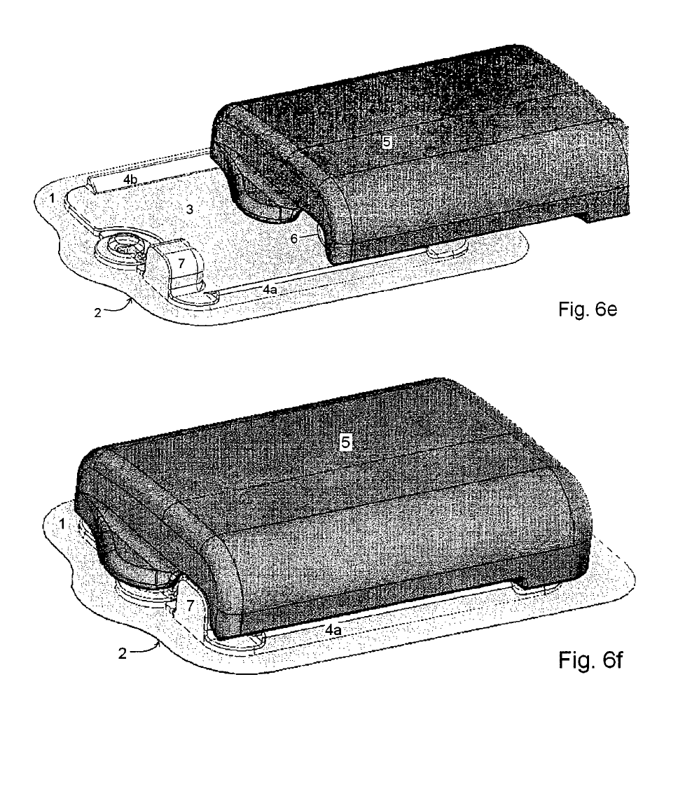

FIG. 6a-6f show a third embodiment of a packing/inserter going through the steps of positioning, insertion and putting into work.

FIG. 1 shows a first embodiment of a transcutaneous device comprising a cannula which can be inserted with the inserter of the present invention. The inserter could also be used e.g. for vaccinations where a single dose of medication has to be injected. The transcutaneous device comprises a mounting part 1 having a mounting surface 2 which is not shown on this figure as the mounting surface is turned towards the patient and is in contact with the patient after mounting of the device. The mounting part 1 can be made of a compliant material which is able to adjust to the patients skin surface and the mounting surface can be an adhesive partly or fully covering the lower side of the mounting part i.e. the side of the mounting part 1 which is turned towards the patient during use. According to one embodiment the mounting part 1 comprises adhesive on both the upper and lower side i.e. on both the mounting surface 2 and the opposite surface. Also the mounting part 1 can be provided with portions of softer and more flexible material (shown as a shaded area around the cannula part). The softer and more flexible material can e.g. be TPE.

A plate 3 of a harder and less flexible material is fastened to the top of the mounting part 2. This plate 3 has been provided with two rails 4a and 4b which rails correspond to grooves or hooks in a corresponding cover 5 for the transcutaneous device. The cover 5 can comprise a pump and means for administrating correct amounts of medication. A reservoir 6 can also be attached either directly or indirectly to the plate 3. If the reservoir 6 is attached indirectly to the plate it will be attached to a connector part 7 via a connector needle 8. The connector part 7 provides a fluid path to a cannula 9 and can be made of e.g. PUR or TPE. The cannula 9 is made of a hard material and is provided with a pointed end which end can cut through the skin. A “hard material” is a material which is hard enough to penetrate the skin surface of the patient and maintain its original shape. According to one embodiment the cannula is a steel needle—G29 and 5 mm long. Normally the needle is less than 10 mm long, where the length is the extent beyond the patients skin.

FIG. 2 shows an enlargement of a second embodiment of a part of a transcutaneous device. The cannula 9 comprises a bend needle made of a hard material which could e.g. be either steel or a hard plastic. The connector part 7 is attached to the plate 3 which plate 3 is provided with an area of a soft and flexible material 3a. In the area of the soft and flexible material 3a the plate 3 can e.g. be able to bend along the line formed by the soft material. This soft and flexible material can e.g. be a layer of TPE.

FIGS. 3a and 3b show the cannula part of FIG. 2 positioned on a transcutaneous device. The transcutaneous device is placed in a packing according to an embodiment of the invention. The packing is formed of a hard top 10 combined with a compressible part 10a, such a packing can be made of more than one material but alternatively the different characteristics of the parts 10 and 10a can also be achieved by varying the form of the material i.e. the material can be corrugated in the compressible part 10a or by varying the thickness of the material i.e. the material can be thinner in the compressible part 10a. A suitable material for the packing would e.g. be PP. FIG. 3a shows the transcutaneous device and the packing the positions they have before insertion and FIG. 3b shows the two units in the positions they have after insertion of the cannula.

Before insertion the packing forms a closed sterile room in which the transcutaneous device is kept, the sterile room is formed as the top 10, 10a of the packing is joined air tight to the mounting part 1.

The packing 10 of the shown embodiment comprises an area of corrugated material 10a which area can be compressed in the direction of insertion i.e. the size of the packing 10 is reduced in this direction. In areas where the packing 10 is not corrugated it is made of a material and in a shape which assures that the packing does not change its dimension during normal use and storing. When a user intends to use the device, the user first removes a release liner 11 which release liner 11 protects the adhesive mounting surface 2 which is fasten the transcutaneous device to the patient during use, then the user pushes the packing 10 down toward the skin of the patient. When the packing 10 is pushed toward the skin, a first part of the plate 3 is in contact with the patients skin and in a fixed position while a second part of the plate 3 is lifted away from the patients skin as the plate 3 is bend around an axis a parallel to the portion of flexible material 3a in the plate 3. When the user wants to insert the cannula 9, the user pushes down on the top of the packing 10 as indicated with an arrow. This movement makes the second part of the plate 3 pivot around the axis a and straightens out the plate 3 and the cannula 9 penetrates first the mounting part 1 and then the patients skin. At last the packing 10 is detached from the transcutaneous device and disposed of. As the packing and the inserter is constituted by the same material the amount of waste is reduced compared to similar products were the inserter is a separate unit.

FIG. 4 shows the transcutaneous device of FIG. 1 after the cannula 9 has been inserted transcutaneously in the patients skin. The transcutaneous device is partly covered by a cover which is half way into its end position, in order to bring the cover 5 to its end and working position it has to be pushed forward in the direction of the arrow. The connector needle 8 of the reservoir 6 can be seen below the cover 5 and when the cover 5 is in the working position, the connector needle 8 has penetrated the protective layer covering the inlet of the connector part 7. The device can be used by patients suffering from diabetes, and then the reservoir 6 will contain insulin.

FIGS. 5a and 5b show a second embodiment of an inserter according to the invention. The transcutaneous device cannot be seen as it is fully covered by the packing. According to this embodiment, the packing 10 has the form of a box where the corrugated part 10a of the packing 10 is placed along the sides of box. When the inserter-packing has been placed on the patients skin and is to be activated, the user pushes the protective top down and thereby causes the not shown cannula of the transcutaneous device to be inserted. This embodiment of the inserter can be made as a two component injection molded package.

FIG. 6a-6f show a third embodiment of an inserter according to the invention. The reference numbers refer to the same parts as in the earlier described embodiments

FIG. 6a shows the closed packing provided with a release liner 11 before use.

FIG. 6b shows the closed packing without release liner 11 ready for use.

FIG. 6c shows the closed packing after insertion of the hard cannula 9.

FIG. 6d shows the transcutaneous device after insertion of the cannula 9 and after the packing/inserter has been removed.

FIG. 6e shows the transcutaneous device in a position where the cover—in this case provided with a pump and controller and a fluid reservoir 6—is half way in end position.

FIG. 6f shows the transcutaneous device in its final working position where fluid is delivered to the patient.

1. A device comprising:
a transcutaneous device comprising:
a mounting part having a mounting surface,
a hard cannula
a fluid path leading fluid from a fluid source to the hard cannula, and
an inserter the inserter comprising a packing and before insertion of the transcutaneous device, the packing forms a closed sterile room covering the transcutaneous device, the packing comprising:
a non-deformable portion which cannot be deformed by the user during normal use and
a deformable portion which can be reduced lengthwise in the direction of insertion during use.

2. The device according to claim 1, wherein the mounting surface is at least partly adhesive and the mounting surface is covered with a release liner before use.

3. The device according to claim 2, wherein the inserter or packing is constructed such that the inserter or the packing can be detached from the transcutaneous device after insertion.

4. The device according to claim 3, wherein the release liner comprises a part of the packing covering an adhesive surface of the transcutaneous device before use.

5. The device according to claim 1, wherein a hard plate is attached to the mounting part.

6. The device according to claim 5, wherein the hard plate comprises a linear part of flexible material dividing the hard plate into a first part and a second part where the second part is fixed to the mounting part and the first part can pivot around an axis a formed by the linear flexible material.

7. The device according to claim 6, wherein an angle α between the first part and the second part in a first position is smaller than 90 degrees and larger than an angle defined by the length of the hard cannula which is to be inserted.

8. The device according to claim 1, wherein the mounting part is provided with a flexible part around an insertion point of the cannula.

9. The device according to claim 1, wherein the hard cannula is made of steel or hard plastic.

10. The device according to claim 1, wherein the fluid source is a reservoir attached to the mounting part.

11. The device according to claim 1, wherein the packing is formed of a hard deformable plastic.

12. The device according to claim 1, wherein the mounting part is provided with two at least partly adhesive surfaces, one surface facing the inserter packing and the mounting surface.

13. The device according to claim 10, wherein the reservoir contains insulin.

14. The device according to claim 11, wherein the packing is formed of two or more types of hard plastic having different ability to be compressed.

15. The device according to claim 1, wherein the deformable portion comprises corrugated material that is compressible in the direction of insertion.
